(19) United States
(12) Patent Application Publication
(10) Pub. No.: US 2020/0268827 A1
(43) Pub. Date: Aug. 27, 2020

(54) COMPOSITION FOR PREVENTION, IMPROVEMENT OR TREATMENT OF OSTEOARTHRITIS COMPRISING ALPINIA OXYPHYLLA EXTRACT AS EFFECTIVE COMPONENT

(71) Applicant: KOREA INSTITUTE OF ORIENTAL MEDICINE, Daejeon (KR)

(72) Inventors: Dong Seon KIM, Daejeon (KR); Yun Mi LEE, Daejeon (KR); Eun Jung SON, Sejong-si (KR); Ji Youn KIM, Daejeon (KR)

(73) Assignee: KOREA INSTITUTE OF ORIENTAL MEDICINE, Daejeon (KR)

(21) Appl. No.: 16/646,387

(22) PCT Filed: Sep. 12, 2018
(86) PCT No.: PCT/KR2018/010706

§ 371 Date: Mar. 11, 2020

(30) Foreign Application Priority Data
Sep. 14, 2017 (KR) 10-2017-0117660

(51) Int. Cl. A61K 36/9062; A61P 19/02

(52) U.S. Cl. CPC A61K 36/9062; A61K 2236/333; A61P 19/02

(57) ABSTRACT

A method for treating osteoarthritis according to an embodiment of the present invention includes administering to a subject in need thereof a composition comprising an extract of Alpinia oxyphylla. The extract of Alpinia oxyphylla can reduce the expression of IL-1β, IL-6, TNF-α and COX-2 as an inflammatory factor and also the expression of MMP-9 as a cartilage degeneration-inducing factor, and can heal the cartilage damage, and thus it can be advantageously used as a functional health food composition for prevention or improvement of osteoarthritis or a therapeutic agent for osteoarthritis.

### TECHNICAL FIELD

The present invention relates to a composition for prevention, improvement, or treatment of osteoarthritis comprising Alpinia oxyphylla extract as an effective component.

### BACKGROUND ART

Arthritis is a disease caused by an occurrence of inflammation and pain in joints, and it includes osteoarthritis, rheumatoid arthritis, gout, and psoriatic arthritis. About 95% of the arthritis patients suffer from osteoarthritis. Osteoarthritis is a disease showing local degenerative changes as the joint cartilage wears down over time, and it is also referred to as “degenerative arthritis.” Osteoarthritis is a representative degenerative disease that is closely related with aging, and it affects almost 10 to 15% of people worldwide. Specifically, about 60 to 80% of the senior people over 65 years old suffer from osteoarthritis.

The main cause of having osteoarthritis is deeply related with aging or obesity, and the disease occurs more frequently and also more severely in women in older age. Initial symptoms of osteoarthritis include stiffiness in one or two joints accompanying sharp pain. As the disease develops over a long period of time, excessive bone formation around joints, deformation of joints, or the like are caused. According to the onset mechanism of osteoarthritis, pro-inflammatory cytokines such as TNF-α, IL-1β, or IL-6 increase and also secretion of MMPs such as collagenase or stromelysin increases to yield articular cartilage damage.

As a therapy currently employed for clinical treatment of osteoarthritis, pharmaceutical therapeutic agents such as pain reliever, steroidal drugs, or non-steroidal anti-inflammatory drugs, or cartilage protective drugs such as hyaluronic acid, glucosamine, or chondroitin are used, or a surgical treatment such as arthroscopic surgery, wedge high tibial osteotomy, hemiarthroplasty, or total knee arthoplasty is carried out. However, the pharmaceutical therapeutic agents have an effect of non-specific alleviation of pain or inflammation itself only, and cartilage protective drugs can only protect joints by supplying nutrients to cartilage cells or absorbing shocks. Furthermore, if steroidal drugs are administered for a long period of time, side effects of having high blood pressure, diabetes, or osteoporosis due to calcium loss are caused. As such, the pharmaceutical therapy has been mostly used for reducing pains, and, although permanent artificial joint replacement is mainly performed, currently no pharmaceuticals or surgical methods are available for providing a basic therapeutic effect.

Meanwhile, Alpinia oxyphylla Fructus is a fruit of Alpinia oxyphylla Miq., which belongs to Zingiberaceae family, and it naturally grows in Haenamsung and Kawndong area in China. Alpinia oxyphylla has both ends with slightly sharp globular or elliptical shape with length of 1 to 2 cm and diameter of 7 to 10 mm. The outside has brown to dark brown color and has several small bump-like protruded lines that are longitudinally connected to each other. Thickness of the fruit skin is 0.3 to 0.5 mm, and, as the fruit skin is tightly adhered to a seed lump, it is difficult to peel the skin. Inside of the fruit is divided lengthwise into three sections by thin membrane, and 5 to 8 seeds, which adhere to one another via a pseudo seed coat, are present in each section. The seed has brown to dark brown color, and, as an irregular polyhedron, it has diameter of about 3.5 mm and hard texture. It has unique smell and slightly bitter taste. As known components of Alpinia oxyphylla, it is believed that nootkatone, epinootkatol, β-nootkatol, β-pinene, p-cymene, terpinen-4-ol, yakuchinone A and B, which are a diarylheptanoid compound, and tectochrysin, chrysin, izalpinin, and 3,5-dihydroxy-7,4′-dimethoxyflavone are contained as a flavonoid component. Nootkatone has an anti-stomach ulcer activity, and yakuchinone A and B are known to have an anti-inflammation activity and can suppress an occurrence of skin cancer and lower the expression of COX-2 and iNOS and the activity of NFκB. Other than those, it is also known that yakuchinone A, nootkatone, and epinootkatol have an insecticidal effect, and they are also known to exhibit an activity of protecting brain cells, an anti-allergy activity, a skin whitening activity, or the like.

It is also known that essential oil components of Alpinia oxyphylla can increase the skin permeability of pharmaceuticals, exhibit an activity of relaxing smooth muscle and suppressing cardiac muscle by suppressing competitively the introduction of calcium ions into a cell, and also have an anti-diuretic activity, an anti-ulcer activity, an anti-dementia activity, and an activity of improving learning abilities.

In Korean Patent Application Publication No. 2012-0109140, a composition and a functional health food product for treating lung cancer comprising an extract of Alpinia oxyphylla are disclosed, and, in Korean Patent Registration No. 1235238, an extract comprising Paeonia radix alba having excellent therapeutic effect for degenerative arthritis and a pharmaceutical composition comprising the extract are disclosed.

However, no descriptions are included in those documents regarding a composition for prevention, improvement, or treatment of osteoarthritis comprising Alpinia oxyphylla extract as an effective component of the present invention.

### DETAILED DESCRIPTION OF THE INVENTION

### Technical Problems to be Solved

The present invention is devised under the circumstances described above, and according to the present invention, a composition for prevention, improvement, or treatment of osteoarthritis comprising an extract of Alpinia oxyphylla as an effective component is provided. Furthermore, by finding that the extract of Alpinia oxyphylla as an effective component of the present invention can reduce the expression of inflammatory factors and MMP-9, which causes articular cartilage damage, and heal the cartilage damage in an animal model, the inventors completed the present invention.

### Technical Means for Solving the Problems

To achieve the object described above, the present invention provides a functional health food composition for prevention or improvement of osteoarthritis comprising an extract of Alpinia oxyphylla as an effective component.

The present invention further provides a pharmaceutical composition for prevention or treatment of osteoarthritis comprising an extract of Alpinia oxyphylla as an effective component.

### Advantageous Effect of the Invention

The present invention relates to a composition for prevention, improvement, or treatment of osteoarthritis comprising an extract of Alpinia oxyphylla as an effective component. Being a material derived from natural products, the extract of Alpinia oxyphylla is safe, has easy availability of raw materials, and has an excellent effect of reducing the expression of IL-1β, IL-6, TNF-α and COX-2 as an inflammatory factor and also the expression of MMP-9 as a cartilage degeneration-inducing factor, and an excellent effect of healing cartilage damage, and thus it can be advantageously used as a raw material of a functional health food composition for prevention or improvement of osteoarthritis or a pharmaceutical composition for prevention or treatment of osteoarthritis.

### BRIEF DESCRIPTION OF THE DRAWINGS

FIG. 1 shows the result of determining percentage of bodyweight loading (%) of hind limb of a SD rat. Con represents a negative control group, MIA (monosodium iodoacetate) represents an MIA-induced osteoarthritis group, MIA+indomethacin represents a positive control group, MIA+Alpinia oxyphylla 300 mg/kg represents a group treated with both MIA and the extract of Alpinia oxyphylla at 300 mg/kg, and MIA+Alpinia oxyphylla 150 mg/kg represents a group treated with both MIA and the extract of Alpinia oxyphylla at 150 mg/kg. In the figure, #, ###, and #### indicate that percentage of bodyweight loading of the group having osteoarthritis induced by MIA is lower in a significant sense than the negative control group, in which # means p<0.05, ### means p<0.001, and #### means p<0.0001. Furthermore, *** indicates that percentage of bodyweight loading of the group treated with both MIA and indomethacin is higher than the MIA-induced osteoarthritis group, in which *** means p<0.001.

FIG. 2 shows the result of determining the expression of an inflammatory factor (IL-1β, IL-6, TNF-α and COX-2) and a cartilage degeneration-inducing factor (MMP-9) in an animal model of osteoarthritis in the presence of an extract of Alpinia oxyphylla. Con represents a negative control group, MIA represents an MIA-induced osteoarthritis group, IM represents a positive control group treated with both MIA and indomethacin, 300 represents a group treated with both MIA and the extract of Alpinia oxyphylla at 300 mg/kg, and 150 represents a group treated with both MIA and the extract of Alpinia oxyphylla at 150 mg/kg. In the figure, # and ## indicate that expression of the inflammatory factor and cartilage degeneration-inducing factor has increased in significant sense compared to con as a negative control group, in which # means p<0.05, and ## means p<0.01. Furthermore, * and ** indicate that, as a result of the treatment with indomethacin or an extract of Alpinia oxyphylla, expression of the inflammatory factor and cartilage degeneration-inducing factor, which had been increased by MIA, has decreased in significant sense, in which * means p<0.05 and ** means p<0.01.

FIG. 3 shows the result of determining histopathological changes in animal model of osteoarthritis by H&E staining, in which the changes are caused by an extract of Alpinia oxyphylla. In the figure, SD rat: Normal represents a negative control group, con represents an MIA-induced osteoarthritis group, Alpinia oxyphylla 300 mg/kg represents a group treated with both MIA and the extract of Alpinia oxyphylla at 300 mg/kg, MIA+Alpinia oxyphylla 150 mg/kg represents a group treated with both MIA and the extract of Alpinia oxyphylla at 150 mg/kg, and indomethacin represents a positive control group treated with both MIA and indomethacin.

FIG. 4 shows the result of determining histopathological changes in animal model of osteoarthritis by safranin-O staining, in which the changes are caused by an extract of Alpinia oxyphylla. The red-stained area represents a layer of proteoglycan, which is one of the major components of cartilage. In the figure, SD rat: Normal represents a negative control group, Control represents an MIA-induced osteoarthritis group, Alpinia oxyphylla 300 mg/kg represents a group treated with both MIA and the extract of Alpinia oxyphylla at 300 mg/kg, MIA+Alpinia oxyphylla 150 mg/kg represents a group treated with both MIA and the extract of Alpinia oxyphylla at 150 mg/kg, and indomethacin represents a positive control group treated with both MIA and indomethacin.

### BEST MODE(S) FOR CARRYING OUT THE INVENTION

The present invention provides a functional health food composition for prevention or improvement of osteoarthritis comprising an extract of Alpinia oxyphylla as an effective component.

It is preferable that the extract of Alpinia oxyphylla of the present invention is extracted by using C₁-C₄ lower alcohol, water, or a mixture thereof as a solvent. It is more preferably extracted by using ethanol as a solvent, but it is not limited thereto.

According to one embodiment of the present invention, the functional health food composition is preferably prepared in formulation type of a powder, a granule, a pill, a tablet, a capsule, a candy, a syrup, or a drink, but it is not limited thereto.

When the functional health food composition is used as a food additive, the extract of Alpinia oxyphylla can be either directly added or used with other food or food components, and it can be suitably used according to a common method. The mixing amount of effective component can be suitably determined depending on a desired use thereof (i.e., prevention, health promotion, or therapeutic treatment). In general, for producing a food product or a drink, the composition of the present invention is added in an amount of 15 parts by weight or less, and preferably 10 parts by weight of less relative to the raw materials. However, when it is used for a long period time, e.g., for maintaining health or hygiene, or keeping a good health state or the like, the mixing amount can be less than the aforementioned range, and, as there is no problem in terms of safety, the effective component can be also used in an amount that is more than the aforementioned range.

Type of the food product is not particularly limited. As for an example of the food products to which the extract or a fraction thereof can be added, it can be any one selected from meats, sausages, breads, chocolates, candies, snacks, cookies, pizza, ramen, other noodles, gums, dairy products including ice cream, various soups, beverages, tea, drinks, alcohol beverages, and vitamin complexes, and it includes any health food products in general sense.

When the composition of the present invention is consumed as a health drink, various flavors or natural carbohydrates may be further included as an additional component like common drinks. Examples of the natural carbohydrates include monosaccharides such as glucose or fructose, disaccharides such as maltose or sucrose, polysaccharides such as dextrin or cyclodextrin, and sugar alcohols such as xylitol, sorbitol, or erythritol. As a sweetening agent, a natural sweetening agent such as taumatin or stevia extract and a synthetic sweetening agent such as saccharine or aspartame can be used. The ratio of the natural carbohydrates is generally about 0.01 to 0.04 g, and preferably about 0.02 to 0.03 g per 100 g of the composition of the present invention. Other than those described in the above, the composition of the present invention may further comprise various nutritional supplements, a vitamin, an electrolyte, a flavor, a coloring agent, pectinic acid and a salt thereof, alginic acid and a salt thereof, an organic acid, protective colloidal thickening agent, a pH adjusting agent, a stabilizer, a preservative, glycerin, alcohol, and a carbonating agent used for carbonated drink. Other than those, fruit flesh for producing natural fruit juice, fruit juice drink, or vegetable drink can be also comprised. Those components may be used either independently or in combination thereof. The ratio of those additives is generally selected, although it is not critical, from a range of 0.01 to 0.1 part by weight per 100 parts by weight of the composition of the present invention.

The present invention also relates to a pharmaceutical composition for prevention or treatment of osteoarthritis comprising an extract of Alpinia oxyphylla as an effective component.

The pharmaceutical composition of the present invention is characterized in that it can reduce, although not limited thereto, the expression of IL-1β, IL-6, TNF-α and COX-2 as an inflammatory factor and also the expression of MMP-9 as a cartilage degeneration-inducing factor.

The composition of the present invention may further comprise, other than the above effective component, a pharmaceutically acceptable carrier, vehicle, or diluent, and can be prepared in various formulations including an oral formulation and a parenteral formulation. In case of producing a formulation, production is made by using a diluent or a vehicle such as filler, bulking agent, binding agent, moisturizing agent, disintegrating agent, or surfactant that are commonly used for producing a formulation. As for the solid formulation for oral administration, a capsule, a powder, a granule, a tablet, a pill or the like are included, and such solid formulation is produced by mixing at least one compound with one or more vehicles such as starch, calcium carbonate, sucrose, lactose, or gelatin. Furthermore, other than simple vehicles, a lubricating agent such as magnesium stearate or talc can be also used. As for the liquid formulation for oral administration, a suspension, an emulsion, a syrup formulation, an aerosol, or the like can be mentioned. Other than water or liquid paraffin as a commonly used simple diluent, various kinds of a vehicle such as moisturizing agent, sweetening agent, aromatic agent, or preservatives may be included. Examples of a formulation for parenteral administration include a sterilized aqueous solution, a non-aqueous formulation, a suspension, an emulsion, a freeze-dried formulation, and a suppository. As a water insoluble solvent or a suspending agent, propylene glycol, polyethylene glycol, or vegetable oil such as olive oil, and injectable ester such as ethylolate can be used. As a base for a suppository, witepsol, macrogol, tween 61, cacao fat, laurin fat, glycerol, gelatin, or the like can be used. In case of parenteral administration, it is preferable to choose external application on skin, intraperitoneal, rectal, intravenous, muscular, subcutaneous, endometrium injection, or intracerebroventricular injection. Most preferably, the composition is used for external application on skin.

The pharmaceutical composition of the present invention is administered in a pharmaceutically effective amount. As described herein, the expression “pharmaceutically effective amount” means an amount sufficient for treating a disorder at reasonable benefit-risk ratio that can be applied for a medical treatment. The effective dose level may be determined based on a type or severeness of a disorder of a patient, activity of a pharmaceutical, sensitivity to a pharmaceutical, administration period, administration route, excretion ratio, time period for therapy, elements including a pharmaceutical used in combination, and other elements that are well known in the medical field. The composition of the present invention can be administered as a separate therapeutic agent, or it can be administered in combination with other therapeutic agent. It can be administered in order or simultaneously with a conventional therapeutic agent. It can be also administered as single-dose or multi-dose. It is important to administer an amount which allows obtainment of the maximum effect with minimum dose while considering all of the aforementioned elements without having any side effect, and the dosage can be easily determined by a person skilled in the pertinent art.

The dosage of the composition of the present invention may vary depending on bodyweight, age, sex, health state, diet of a patient, administration period, administration method, excretion rate, and severeness of disorder. The composition of the present invention may be also used either singly or in combination with a surgery, a radiation therapy, a hormone therapy, a chemotherapy, or a method of using biological response modifier, or the like.

Hereinbelow, the present invention is explained in greater detail in view of the Examples. However, the following Examples are given only for specific explanation of the present invention and it wound be evident to a person who has common knowledge in the pertinent art that the scope of the present invention is not limited by them.

### EXAMPLES

### Example 1. Preparation of Extract of Alpinia oxyphylla

To 1 kg of Alpinia oxyphylla, 15 t of 70% (v/v) ethanol were added, and, after reflux extraction for 3 hours at 85° C., the filtered solution was concentrated under reduced pressure at 50° C. followed by drying to obtain 11.7 g of an extract of Alpinia oxyphylla.

### Example 2. Determination of Effect of Extract of Alpinia oxyphylla on Bodyweight Loading in Animal Model of MIA (Monosodium Iodoacetate)-Induced Osteoarthritis

To determine the effect of an extract of Alpinia oxyphylla, which has been prepared in the above Example 1, on bodyweight loading in an animal model of MIA-induced osteoarthritis, MIA as an osteoarthritis-inducing material (diluted to 60 mg/ml with 0.9% physiological saline) was administered in an amount of 50 μl to an articular cavity of a right hind limb of 7-week old SD rat to induce osteoarthritis. After that, the animal was orally administered with an extract of Alpinia oxyphylla (150 or 300 mg/kg) once a day for 21 days, while the percentage of bodyweight loading was measured every 7 days. As a positive control group, indomethacin (1 mg/kg) was used.

The bodyweight loading on hind limb was measured by using a paw weight tester (Incapacitance tester, Linton instrument Co., UK, Ser No. 01/45/25). In a tester holder, the rat with induced osteoarthritis tends to stand on a normal paw which has not been administered with MIA due to the pain occurring in the other paw, and thus balance between the weights of two paws was not maintained so that the weight of paw administered with MIA is measured to be relatively lighter than the weight of normal paw. For measuring the weight of paw, caution was taken such that the abdomen of SD rat does not touch the sensor of device, and weight (g) of the each paw was measured separately. Then, by using the weight of paw which has been measured accordingly, percentage of bodyweight loading (%) was calculated based on the following Formula 1. The bodyweight loading indicates the pressing force exhibited by paw for maintaining the posture, and in normal cases, percentage of bodyweight loading is 50% for single paw as the weights of two paws are in the balance. However, as the pain increases due to the induced osteoarthritis, lower percentage of bodyweight loading (%) is yielded in the hind limb with induced osteoarthritis.

Percentage of bodyweight loading (%)=(Weight of hind limb with induced arthritis/Weight of both hind limbs)×100 Formula 1

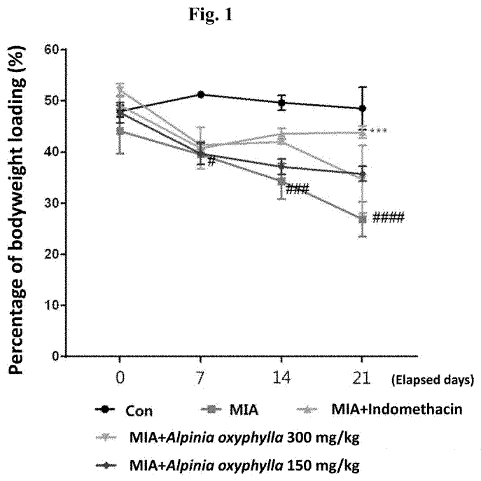

As a result, it was found that, in the group with MIA-induced osteoarthritis, the percentage of bodyweight loading (%) decreased almost by half over time compared to the control group as it is shown in FIG. 1. On the other hand, the group treated with both MIA and an extract of Alpinia oxyphylla showed increased percentage of bodyweight loading (%) compared to the MIA administration group. On Day 21 after the administration of an extract of Alpinia oxyphylla, this increase over the MIA administration group was shown regardless of a difference in the administration concentration, i.e., 150 mg/kg and 300 mg/kg.

### Example 3. Determination of Effect of Extract of Alpinia oxyphylla on Inflammatory Factor and Cartilage Degeneration-Inducing Factor in Animal Model of MIA (Monosodium Iodoacetate)-Induced Osteoarthritis

From a joint with induced osteoarthritis, mRNA was extracted and the expression of an inflammatory factor (IL-1β, IL-6, TNF-α and COX-2) and a cartilage degeneration-inducing factor (MMP-9) was examined by PCR.

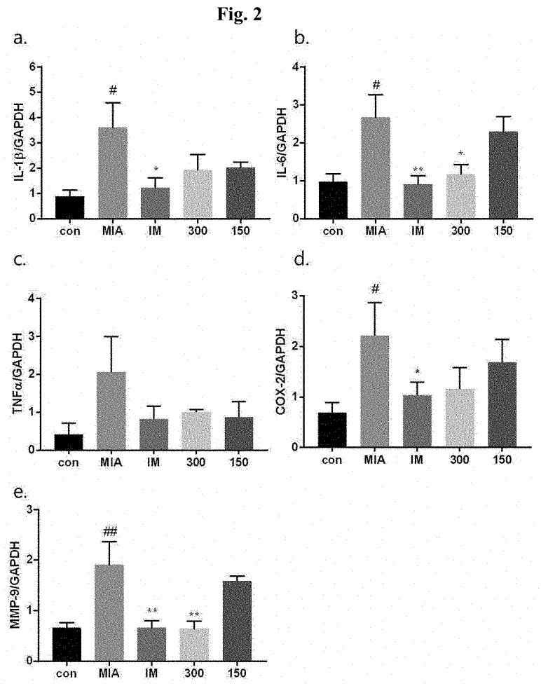

As a result, it was shown that the expression of an inflammatory factor and a cartilage degeneration-inducing factor has increased in a sample with MIA-induced osteoarthritis as it is shown in FIG. 2. On the other hand, when the sample is treated also with an extract of Alpinia oxyphylla, the expression of those increased factors has decreased. Based on this result, it was found that the inflammation and articular cartilage damage caused by osteoarthritis can be suppressed by an extract of Alpinia oxyphylla.

### Example 4. Determination of Histopathological Changes Caused by Extract of Alpinia oxyphylla in Animal Model of MIA (Monosodium Iodoacetate)-Induced Osteoarthritis

The knee part of a rat with induced osteoarthritis was excised and placed in 10% formalin solution containing 10% EDTA to remove calcium from the joint tissues. After that, the joint tissues were added to paraffin wax for fixing, and, by carrying out coronal section, the paraffin-fixed tissues were cut to a size of 7 μm. Then, by performing H&E (hematoxylin and eosin) staining and safranin-O staining, state of the tissues was examined. Presence or absence of an inflammatory response, proliferation of synovial membrane cells, and tissue infiltration by inflammatory cells were determined by H&E staining, and, based on safranin-O staining for staining a layer of proteoglycan, which is one of the major components of cartilage, any damage occurring in cartilage tissues was examined.

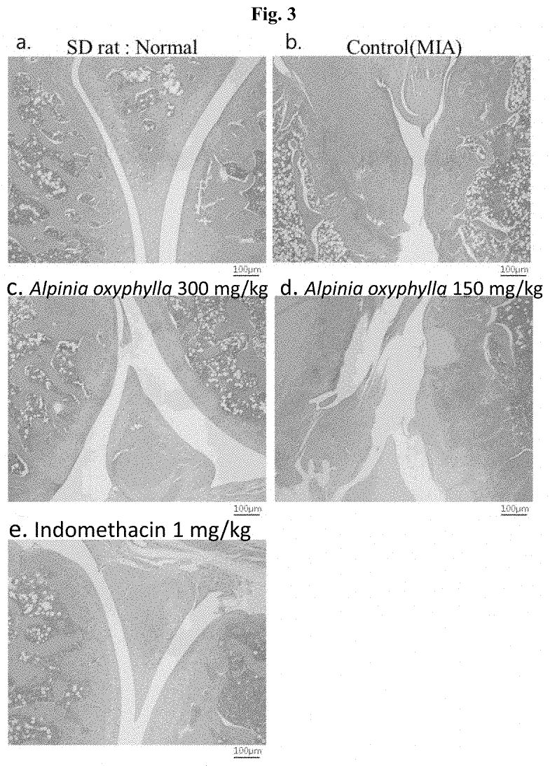

As a result, it was shown that, according to H&E staining, the joint synovial membrane tissues are in a normal position in the normal group (SD rat: Normal), while the group with MIA-induced osteoarthritis (Control (MIA)) shows the loss of synovial membrane tissues caused by infiltration of cartilages and bones, resulting from excessive invasion of synovial membrane cells around the joint. It was also found that such infiltration of cartilages and bones is reduced by administration of an extract of Alpinia oxyphylla as it is shown in FIG. 3.

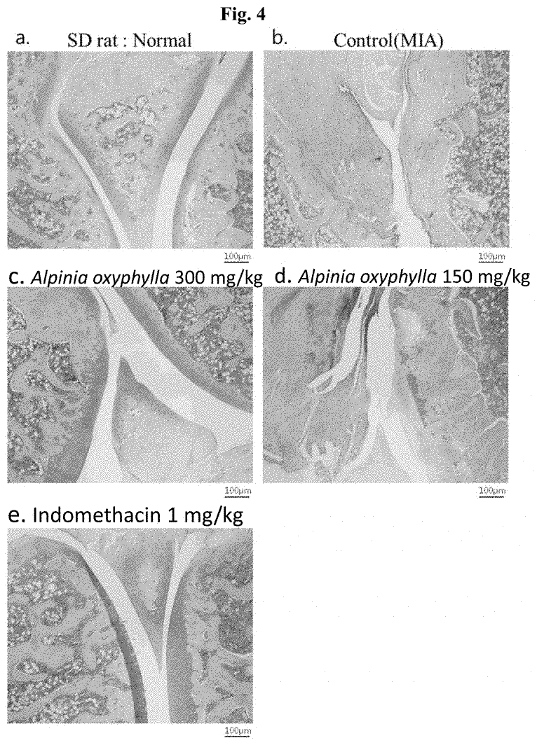

Furthermore, as it is shown in FIG. 4, according to the result of safranin-O staining, the red-stained normal cartilage tissues were damaged by MIA in the group with induced osteoarthritis (Control (MIA)) so that a disappearance of the proteoglycan tissues was yielded. However, when the treatment was carried out together with an extract of Alpinia oxyphylla, the red-stained proteoglycan tissues remained in large amounts around the synovial membrane. Based on this result, it was found that osteoarthritis induced by MIA can be suppressed by an extract of Alpinia oxyphylla.

1-7: (canceled)

8: A method for treating osteoarthritis, the method comprising administering to a subject in need thereof a composition comprising an extract of Alpinia oxyphylla.

9: The method of claim 8, wherein the extract of Alpinia oxyphylla is extracted by using C1-C4 lower alcohol, water, or a mixture thereof as a solvent.

10: The method of claim 8, wherein the composition is in a formulation type selected from the group consisting of a powder, a granule, a pill, a tablet, a capsule, a candy, a syrup, and a drink.

11: The method of claim 8, wherein the composition is a pharmaceutical composition.

12: The method of claim 11, wherein the pharmaceutical composition is in a formulation type selected from the group consisting of a capsule, a powder, a granule, a tablet, and a pill.

13: The method of claim 11, wherein the pharmaceutical composition further comprises a pharmaceutically acceptable carrier, a vehicle, or a diluent.
